(19) United States
(12) Patent Application Publication
(10) Pub. No.: US 2016/0158134 A1
(43) Pub. Date: Jun. 9, 2016

(54) Stable Vitamin C System

(71) Applicant: Jeane Disalvo, Marina del Rey, CA (US)

(72) Inventor: Jeane Disalvo, Marina del Rey, CA (US)

(21) Appl. No.: 14/563,897

(22) Filed: Dec. 8, 2014

(51) Int. Cl. A61K 8/49; A61Q 19/00; A61K 8/98; A61Q 19/08; A61Q 17/04; A61Q 15/00

(52) U.S. Cl. CPC A61K 8/4973; A61Q 17/04; A61Q 15/00; A61K 8/981; A61Q 19/08; A61Q 19/007; A61K 2800/20

(57) ABSTRACT

The present compositions include substantially anhydrous lotions, gels, or cremes having a high vitamin C content. The compositions described herein may be used as paste, lotions, cremes, or gels in and of themselves, or as a solvent/base system for additional ingredients. Additional ingredients may include therapeutically active ingredients, pigments, fragrances or other cosmetic additives.

L-ascorbic acid (Vitamin C) and derivatives thereof are anti-oxidants. Ascorbic acid is very soluble in aqueous media, but aqueous media renders the L-ascorbic acid unstable. For instance, aqueous solutions of the free acid are rapidly oxidized by air; the reaction is accelerated by alkalines, iron, and copper. Similarly, the sodium salt is unstable and subject to quick oxidation by air at a pH>6. Ascorbic acid is only slightly soluble in non-aqueous media (such as absolute ethanol having a solubility of 1 g/50 ml), but is insoluble in ether, chloroform, benzene, petroleum ether, oils, fats, or fat solvents.

Lotions are frequently aqueous mixtures, or oil-in-water (O/W) emulsions. Accordingly, the using large amounts of L-ascorbic acid in an aqueous lotion, gel, or creme results in degraded L-ascorbic acid where much of the therapeutic benefit has been lost. Furthermore, if solid L-ascorbic acid powder is used in a non-aqueous mixture in any therapeutically significant amount, the “feel” of the lotion, gel, or crème is compromised, that is, the lotion, gel, or crème becomes gritty. Accordingly, there is a need for lotions, gels, or cremes containing high amounts of solid L-ascorbic acid (or derivatives thereof) which have a soft texture, glide over the skin smoothly, which are stable for storage, and which provide the anti-oxidant benefits of L-ascorbic acid.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Unstable Vitamin C crème after being stored for three months in an incubator at 45 degrees C. Notably, the cream is a water-in-oil emulsion, such as used by conventional cremes known in the art. As can be seen from the picture, the crème is very discolored, has separated into multiple layers or phases, and has reduced volume. The lower layer is dark brown, the upper layer is a yellow/brown/orange color, and discolored, granulated remnants of the crème adhere to the walls of the container.

FIG. 2: Unstable Vitamin C serum after being stored for three months in an incubator at 45 degrees C. When initially created, this serum was a clear viscous liquid. After three months in the incubator at 45 degrees C., the serum is very discolored, and has taken on a dark honey color.

FIG. 3, FIG. 4, and FIG. 5 are pictures of embodiments of the presently claimed compositions. In particular, FIG. 3 and FIG. 4 show the anhydrous composition of the present invention in cream form, having at least 10% or more vitamin C after three months in the incubator at 45° C. FIG. 5 shows the anhydrous composition of the present invention in cream form having at least 10% or more vitamin C, immediately after being mixed.

### BRIEF SUMMARY OF THE INVENTION

The present invention will be further described in the following non-limiting embodiments.

1. A substantially anhydrous composition comprising:
(a) about 1-40% by weight powdered L-ascorbic acid, or L-ascorbic acid derivatized at the C2, C3, C5, or C6 position;
(b) one or more crosspolymers and/or oleosomes as natural substitute for encapsulating the L-ascorbic acid,
(c) at least one medium to high melting lipid (including ester waxes);
(d) one or more volatile compounds as dispersing agent for L-ascorbic acid.
   2. The substantially anhydrous composition according to embodiment 1, wherein the powdered L-ascorbic acid derivatized at the C2, C3, C5, or C6 position is water-soluble.
   3. The substantially anhydrous composition according to any of embodiments 1-2, wherein the derivative is ethyl-ascorbic acid, ascorbyl-2-glucoside (ASC-G), ascorbyl-6-octanoate (ASC-8), ascorbyl-6-palmitate (ASC-P), ascorbyl-6-stearate (ASC-S), ascorbyl-2,6-dipalmitate (ASC-DP), L-dehydroascrobic acid, sodium ascorbyl phosphate, ascorbyl tetraisopalmitate/tetrahexyldecyl ascorbate, or magnesium ascorbyl phosphate.
   4. The substantially anhydrous composition according to any of embodiments 1-3, comprising about 10-30% by weight L-ascorbic acid, or L-ascorbic acid derivatized at the C2, C3, C5, or C6 position.
   5. The substantially anhydrous composition according to any of embodiments 1-4, comprising about 20-30% by weight L-ascorbic acid, or L-ascorbic acid derivatized at the C2, C3, C5, or C6 position.
   6. The substantially anhydrous composition according to any of embodiments 1-5 wherein the one or more crosspolymers are selected from the group consisting of:
   acrylates/bis-hydroxypropyl dimethicone crosspolymer, behenyl dimethicone/bis-vinyldimethicone crosspolymer; bis-phenylisopropyl phenylisopropyl dimethicone/vinyl dimethicone crosspolymer; bis-vinyldimethicone/bis-isobutyl PPG-20 crosspolymer; bis-vinyldimethicone crosspolymer; bis-vinyldimethicone/PEG-10 dimethicone crosspolymer; bis-vinyldimethicone/PPG-20 crosspolymer; butyldimethicone methacrylate/methyl methacrylate crosspolymer; C30-45 alkyl cetearyl dimethicone crosspolymer; C4-24 alkyl dimethicone/divinyldimethicone crosspolymer; C30-45 alkyl dimethicone/polycyclohexene oxide crosspolymer; cetearyl dimethicone crosspolymer; cetearyl dimethicone/vinyl dimethicone crosspolymer; cetyl dimethicone/bis-vinyldimethicone crosspolymer; cetyl hexacosyl dimethicone/bis-vinyldimethicone crosspolymer; crotonic acid/vinyl C8-12 isoalkyl esters/VA/bis-vinyldimethicone crosspolymer; dimethicone/bis-isobutyl PPG-20 crosspolymer; dimethicone/bis-vinyldimethicone/silsesquioxane crosspolymer; dimethicone crosspolymer; dimethicone crosspolymer-3; dimethicone/divinyldimethicone/silsesquioxane crosspolymer; dimethicone/lauryl dimethicone/bis-vinyldimethicone crosspolymer; dimethicone/PEG-10 crosspolymer; dimethicone/PEG-10/15 crosspolymer; dimethicone/PEG-15 crosspolymer; dimethicone/phenyl vinyl dimethicone crosspolymer; dimethicone/polyglycerin-3 crosspolymer; dimethicone/PPG-20 crosspolymer; dimethicone/titanate crosspolymer; dimethicone/vinyl dimethicone crosspolymer; dimethicone/vinyltrimethylsiloxysilicate crosspolymer; diphenyl dimethicone crosspolymer; diphenyl dimethicone/vinyl diphenyl dimethicone/silsesquioxane crosspolymer; divinyldimethicone/dimethicone crosspolymer; hydroxypropyl dimethicone/polysorbate 20 crosspolymer; isopropyl titanium triisostearate/triethoxysilylethyl polydimethylsiloxyethyl dimethicone crosspolymer; lauryl dimethicone PEG-15 crosspolymer; lauryl dimethicone/polyglycerin-3 crosspolymer; lauryl polydimethylsiloxyethyl dimethicone/bis-vinyldimethicone crosspolymer; PEG-10 dimethicone crosspolymer; PEG-12 dimethicone crosspolymer; PEG-8 dimethicone/polysorbate 20 crosspolymer; PEG-12 dimethicone/bis-isobutyl PPG-20 crosspolymer; PEG-12 dimethicone/PPG-20 crosspolymer; PEG-10 dimethicone/vinyl dimethicone crosspolymer; PEG-10/lauryl dimethicone crosspolymer; PEG-15/lauryl dimethicone crosspolymer; PEG-15/lauryl polydimethylsiloxyethyl dimethicone crosspolymer; perfluorononyl dimethicone/methicone/amodimethicone crosspolymer; polydimethylsiloxyethyl dimethicone/bis-vinyldimethicone crosspolymer; polyglyceryl-3/lauryl polydimethylsiloxyethyl dimethicone crosspolymer; silicone quatemium-16/glycidoxy dimethicone crosspolymer; styrene/acrylates/dimethicone acrylate crosspolymer; trifluoropropyl dimethicone/PEG-10 crosspolymer; trifluoropropyl dimethicone/trifluoropropyl, divinyldimethicone crosspolymer; trifluoropropyl dimethicone/vinyl trifluoropropyl dimethicone/silsesquioxane crosspolymer; trimethylsiloxysilicate/dimethicone crosspolymer; vinyl dimethicone/lauryl/behenyl dimethicone crosspolymer; vinyl dimethicone/lauryl dimethicone crosspolymer; vinyl dimethicone/methicone silsesquioxane crosspolymer; vinyldimethyltrimethylsiloxysilicate/dimethicone crosspolymer; and vinyldimethyl/trimethylsiloxysilicate stearyl dimethicone crosspolymer.
   7. The substantially anhydrous composition according to any of embodiments 1-6, wherein the crosspolymer is dimethicone cross polymer having a molecular weight ranging from about 15,000-1,000,000.
   8. The substantially anhydrous composition according to embodiment 6, wherein the one or more crosspolymers are selected from the group consisting of: dimethicone and dimethicone/vinyl dimethicone crosspolymer.
   9. The substantially anhydrous composition according to any one of embodiments 1-8, wherein the amount of crosspolymer ranges from about 5% by weight to 60% by weight.
   10. The substantially anhydrous composition according to any of embodiments 1-9, wherein the amount of crosspolymer ranges from about 10% to 60%.
   11. The substantially anhydrous composition according to any of embodiments 1-10, wherein the amount of crosspolymer ranges from about 30% to 50%.
   12. The substantially anhydrous composition according to any of embodiments 1-11, wherein the high melting point lipid has a melting point ranging from about 30° C. to about 80° C.
   13. The substantially anhydrous composition according to any of embodiments 1-12, wherein the medium to high melting lipid is selected from the group consisting of: shea butter, apricot butter, cocoa butter, mango butter, kokum butter, illipe butter, cupuacu butter, murumuru seed butter, acrocomia aculeata butter, astrocaryum tucuma butter, attalea maripa butter, bassia latifolia seed butter, camellia reticulate seed butter, carpotroche brasiliensis seed butter, coconut seed butter, dipteryx odorate seed butter, palm butter, strawberry seed butter, sunflower seed butter, grcinia indica seed butter, walnut seed butter, irvingia gabonensis kemel butter, sesame seed butter, avocado butter, pentadesma butyracea seed butter, jojoba butter, shorea robusta seed butter, Theobroma grandiflorum seed butter, trichilia seed butter, virola surinamensis seed butter, pouteria sapota seed butter, tribehenin.
   14. The substantially anhydrous composition according to any of the preceding embodiments wherein the amount of high melting point lipid ranges from 1% to 30%.
   15. The substantially anhydrous composition according to any of the preceding embodiments wherein the amount of high melting lipid at most 5%.
   16. The substantially anhydrous composition according to any of embodiments 1-15, wherein the composition further comprises one or more low melting point lipid(s). In one aspect of this embodiment, the low melting point lipid(s) is selected from an oil selected from one or more of the group consisting of cyclopentasiloxane, olus oil, camelina sativa oil, vegetable oil, palm oil, camelina sativa seed oil, Abyssinian oil, apricot kernel oil, fractionated coconut oil, jojoba oil, mongongo nut oil, pine nut oil, cashew oil, beech nut oil, sweet almond oil, walnut oil, wheat germ oil, avocado oil, borage seed oil, cranberry seed oil, evening primrose oil, grape seed oil, hazelnut oil, kukui nut oil, macademia nut oil, meadowfoam oil, olive oil, peanut oil, pecan oil, pomegranate seed oil, rose hip oil, seabuckthom berry oil, sesame seed oil, sunflower oil, watermelon seed oil, canoia oil, camellia oil, tigemut oil, thistle oil, safflower oil, oat oil, marula oil, Moroccan oil, dika oil, fish oil, kapok seed oil, peanut oil, seje oil, sacha inchi oil, palm kemel oil, kenaf seed oil, and argan oil.
   17. The substantially anhydrous composition according to any of embodiments 1-16, wherein the medium to high melting point lipid or lipid ester is a wax selected from one or more of the group consisting of: Rhus Succedanea Fruit Wax, Polianthes Tuberosa Flower Wax, Helianthus Annuus (Sunflower) Seed Wax, Rosa Multiflora Flower Wax, Citrus Aurantium Dulcis (Orange) Flower Wax, Narcissus Poeticus Flower Wax, Acacia Decurrens Flower Wax, Jasminum Grandiflorum (Jasmine) Flower Wax, Nelumbo Nucifera Flower Wax, Candelilla Cera, Cistus Ladaniferus Flower Wax, Citrus Aurantium Amara (Bitter Orange), Cananga Odorata Flower Wax, Citrus Limon (Lemon) Peel Wax, Copemicia Cerifera (Camauba) Wax, Copemicia Cerifera Cera, Euphorbia Cerifera (Candelilla) Wax, Cytisus Scoparius Flower Wax, Myrica Cerifera (Bayberry) Fruit Wax, Tuberose Wax, Palm Kernel Wax, Helichrysum Angustifolium Wax, Ericerus Pela Wax, and Tribehenin. Animal derived waxes are also contemplated, including, without limitation: Beeswax, Lanolin Wax, or Petrolatum wax.
   18. The substantially anhydrous composition according to any of embodiments 1-17, wherein the amount of volatile compound(s) ranges from 1% to 60% by weight.
   19. The substantially anhydrous composition according to any of embodiments 1-18, wherein the amount of volatile compound(s) at least 5% by weight.
   20. The substantially anhydrous composition according to any of embodiments 1-19, wherein the volatile compound(s) is a silicon containing volatile oil.
   21. The substantially anhydrous composition according to any of embodiments 1-20, wherein the volatile compound(s) is a siloxane, a silicon-containing volatile oil, or a hydrocarbon,
   22. The substantially andhydrous composition according to embodiment 21, wherein the hydrocarbon is isododecane, undecane, tridecane, coconut alkanes.
   23. The substantially anhydrous composition according to any of embodiments 1-22, wherein the volatile compound(s) is selected from the group consisting of, diolyldimethylsiloxane, cyclopentylsiloxane, cyclomethicone, polymethylphenylsilxoxane.
   24. The substantially anhydrous composition according to any of embodiments 1-23 in the form of an oil, gel, paste, lotion, crème, or aerosolized oil, gel, lotion or crème.
   25. In a further embodiment, the composition comprises a substantially anhydrous composition comprising:
(a) about 1-40% by weight powdered L-ascorbic acid, or L-ascorbic acid derivatized at the C2, C3, C5, or C6 position;
(b) one or more crosspolymers,
(c) at least one low melting point lipid;
(d) one or more volatile compounds.
   26. The substantially anhydrous composition according to embodiment 25 in the form of a gel, where the meaning of the ingredients (a)-(d) is consistent with the various ingredients and combinations of ingredients of any of embodiments 1-24.
   27. The substantially anhydrous composition of any one of embodiment 1 to 26 wherein the composition further comprises one or more additional active ingredients.
   28. The substantially anhydrous composition of embodiment 27, wherein the additional active ingredient is selected from an antiperspirant, a sunscreen, a yeast extract, a probiotic mixture, a stem cell mixture, or an antibiotic.
   29. The substantially anhydrous composition of embodiment 27, wherein the antiperspirant is selected from aluminum chlorohydrate, aluminum zirconium tetrachlorohydrex, aluminum sulfate, ferric chloride hexahydrate, zirconium, and zirconyl chloride octahydrate.
   30. The substantially anhydrous composition of embodiment 28, wherein the sunscreen is selected from one or more of zinc oxide, titanium dioxide, oxybenzone, 3-(4-methylbenzylidene)-camphor (4-MBC), octyl-methoxycinnamate (OMC), octyl-dimethyl-PABA (OD-PABA), bexophenome-3 (Bp-3) and homosalate.
   31. The substantially anhydrous composition of embodiment 28, wherein the antibiotic is one or more of a sulfonamide, an aminoglycoside, a quinolone, a oxazoladinone, gramicidin, or a tyrocidine.
   32. The substantially anhydrous composition of any of embodiments 1-31, wherein the additional active ingredient is steviocide, tea tree oil, retinoic acid, Tocotrienols or Vitamin E, Vitamin A, hyaluronic acid and derivatives thereof.
   33. In one embodiment, the present invention includes A method for treating skin wrinkles, for skin lightening, for increasing collagen production or retention, for moisturizing skin, for treating or alleviating acne, or for preventing sun damage comprising administering the composition of any one of embodiment 1-32.

Further embodiments contemplated herein will be clear to the skilled artisan. Further description of the various embodiments is provided below. The skilled artisan would recognize that combinations of particular ingredients and variations are contemplated.

### DETAILED DESCRIPTION

The present compositions include substantially anhydrous paste, gels, or cremes having a high vitamin C content. The compositions described herein may be used as lotions, creams (aka “cremes”), or gels in and of themselves, or as a solvent/base system for additional ingredients. Additional ingredients may include therapeutically active ingredients, pigments, fragrances or other cosmetic additives.

Vitamin C and L-ascorbic acid are used herein interchangeably. Derivatives of L-ascorbic acid include L-ascorbic acid derivatized (modified by the substitution, addition, or deletion of a functional group) at the C2, C3, C5, or C6 position. In one embodiment, ethyl-ascorbic acid, ascorbyl-2-glucoside (ASC-G), ascorbyl-6-octanoate (ASC-8), ascorbyl-6-palmitate (ASC-P), ascorbyl-6-stearate (ASC-S), ascorbyl-2,6-dipalmitate (ASC-DP), L-dehydroascrobic acid, sodium ascorbyl phosphate, potassium ascrobyl phosphate, tetrahexaldecyl ascorbate, ascorbyl tetraisopalmitate/tetrahexyldecyl ascorbate, or magnesium ascorbyl phosphate. Furthermore, vitamin C may be obtained from vitamin C rich plant extracts. Such vitamin C rich plant extracts include Malpighia Glabra (Acerola) Fruit Extract and Terminalia Ferdinandiana Fruit Extract. In addition, extracts may be obtained from Vitamin C-rich fruits (or extracts therefrom), which include, without limitation, Papaya, kiwi, strawberry, and orange.

In one embodiment, the L-ascorbic acid or derivative thereof is a powdered solid. In an aspect of this embodiment, the L-ascorbic acid or derivative thereof does not dissolve significantly into the substantially anhydrous mixture of ingredients used in the present composition. In one aspect of this embodiment, the powdered L-ascorbic acid or derivative thereof is an ultra fine powder having an average particle size ranging from less than or equal to 100 microns, less than or equal to 75 microns, less than or equal to 50 microns, o less than or equal to 25 microns, less than or equal to 10 microns, or less than or equal to 5 microns. In one aspect, the present compositions include powdered L-ascorbic acid or a derivative thereof having an average particle size ranging from about 5 to 100 microns, from about 5 to 50 microns, from about 5 to 25 microns. In another aspect, the present compositions include powdered L-ascorbic acid or a derivative thereof having a particle size ranging from 5 to 100 microns, from 10 to 100 microns, from 10 to 50 microns, from 10 to 75 microns, from 10 to 25 microns, or from 5 to 10 microns.

The high vitamin C content of the present compositions includes compositions having a high percentage of vitamin c by weight. In one embodiment, the present compositions have at least about 10%, at least about 11%, at least about 12%, at least about 13% at least about 14%, at least about 15%, at least about 16% at least about 17%, at least about 18%, at least about 19% at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 42%, at least about 45%, at least about 47%, or at least about 50% by weight powdered ascorbic acid or a derivative thereof. In one embodiment, the present compositions have from about 10% to 55% by weight, 10% to 40% by weight, 10% to 30% by weight, 15% to 55% by weight, 15% to 50% by weight, 15% to 45% by weight, 15% to 40% by weight, 15% to 30% by weight, 15% to 25% by weight, 20% to 55% by weight, 20% to 50% by weight, 20% to 45% by weight, 20% to 40% by weight, 20% to 35% by weight, 20% to 30% by weight, 25% to 55% by weight, 25% to 50% by weight, 25% to 45% by weight, 25% to 40% by weight, 25% to 35% by weight, 25% to 30% by weight, 30% to 55% by weight, 30% to 50% by weight, 30% to 45% by weight, 30% to 40% by weight, 30% to 45% by weight, 35% to 55% by weight, 35% to 50% by weight, 35% to 45% by weight, 35% 40% by weight, 40% to 55% by weight, 40% to 50% by weight, 40% to 45% by weight powdered ascorbic acid or a derivative thereof. In one embodiment, the amount of ascorbic acid is at least 30% by weight.

In one embodiment, the cross-polymers or oleosomes partially or wholly encapsulate the vitamin C or L-ascorbic acid derivative. This encapsulation provides a barrier between the vitamin C and the other anhydrous components of the composition, maintaining stability and preventing oxidation of the vitamin C or spoilage of the other ingredients.

The crosspolymers described herein include, without limitation, acrylates/bis-hydroxypropyl dimethicone crosspolymer, behenyl dimethicone/bis-vinyldimethicone crosspolymer; bis-phenylisopropyl phenylisopropyl dimethicone/vinyl dimethicone crosspolymer; bis-vinyldimethicone/bis-isobutyl PPG-20 crosspolymer; bis-vinyldimethicone crosspolymer; bis-vinyldimethicone/PEG-10 dimethicone crosspolymer; bis-vinyldimethicone/PPG-20 crosspolymer; butyldimethicone methacrylate/methyl methacrylate crosspolymer; C30-45 alkyl cetearyl dimethicone crosspolymer; C4-24 alkyl dimethicone/divinyldimethicone crosspolymer; C30-45 alkyl dimethicone/polycyclohexene oxide crosspolymer; cetearyl dimethicone crosspolymer; cetearyl dimethicone/vinyl dimethicone crosspolymer; cetyl dimethicone/bis-vinyldimethicone crosspolymer; cetyl hexacosyl dimethicone/bis-vinyldimethicone crosspolymer; crotonic acid/vinyl C8-12 isoalkyl esters/VA/bis-vinyldimethicone crosspolymer; dimethicone/bis-isobutyl PPG-20 crosspolymer; dimethicone/bis-vinyldimethicone/silsesquioxane crosspolymer; dimethicone crosspolymer; dimethicone crosspolymer-3; dimethicone/divinyldimethicone/silsesquioxane crosspolymer; dimethicone/lauryl dimethicone/bis-vinyldimethicone crosspolymer; dimethicone/PEG-10 crosspolymer; dimethicone/PEG-10/15 crosspolymer; dimethicone/PEG-15 crosspolymer; dimethicone/phenyl vinyl dimethicone crosspolymer; dimethicone/polyglycerin-3 crosspolymer; dimethicone/PPG-20 crosspolymer; dimethicone/titanate crosspolymer; dimethicone/vinyl dimethicone crosspolymer; dimethicone/vinyltrimethylsiloxysilicate crosspolymer; diphenyl dimethicone crosspolymer; diphenyl dimethicone/vinyl diphenyl dimethicone/silsesquioxane crosspolymer; divinyldimethicone/dimethicone crosspolymer; hydroxypropyl dimethicone/polysorbate 20 crosspolymer; isopropyl titanium triisostearate/triethoxysilylethyl polydimethylsiloxyethyl dimethicone crosspolymer; lauryl dimethicone PEG-15 crosspolymer; lauryl dimethicone/polyglycerin-3 crosspolymer; lauryl polydimethylsiloxyethyl dimethicone/bis-vinyldimethicone crosspolymer; PEG-10 dimethicone crosspolymer; PEG-12 dimethicone crosspolymer; PEG-8 dimethicone/polysorbate 20 crosspolymer; PEG-12 dimethicone/bis-isobutyl PPG-20 crosspolymer; PEG-12 dimethicone/PPG-20 crosspolymer; PEG-10 dimethicone/vinyl dimethicone crosspolymer; PEG-10/lauryl dimethicone crosspolymer; PEG-15/lauryl dimethicone crosspolymer; PEG-15/lauryl polydimethylsiloxyethyl dimethicone crosspolymer; perfluorononyl dimethicone/methicone/amodimethicone crosspolymer; polydimethylsiloxyethyl dimethicone/bis-vinyldimethicone crosspolymer; polyglyceryl-3/lauryl polydimethylsiloxyethyl dimethicone crosspolymer; silicone quatemium-16/glycidoxy dimethicone crosspolymer; styrene/acrylates/dimethicone acrylate crosspolymer; trifluoropropyl dimethicone/PEG-10 crosspolymer; trifluoropropyl dimethicone/trifluoropropyl, divinyldimethicone crosspolymer; trifluoropropyl dimethicone/vinyl trifluoropropyl dimethicone/silsesquioxane crosspolymer; trimethylsiloxysilicate/dimethicone crosspolymer; vinyl dimethicone/llauryl/behenyl dimethicone crosspolymer; vinyl dimethicone/lauryl dimethicone crosspolymer; vinyl dimethicone/methicone silsesquioxane crosspolymer; vinyldimethyl/trimethylsiloxysilicate/dimethicone crosspolymer; and vinyldimethyl/trimethylsiloxysilicate stearyl dimethicone crosspolymer.

In one embodiment, the crosspolymer is dimethicone crosspolymer. In one embodiment, the crosspolymer is dimethicone crosspolymer having a molecular weight ranging from about 15,000-1,000,000.

The amount of crosspolymer generally is around at most 60%, at most 50%, at most 40%, at most 30%, at most 25%, at most 20%, at most 15%, or at most 10%. In one embodiment, the amount of crosspolymer ranges from 5% to 60%, from 10% to 60%, from 20% to 60%, from 25% to 60%, from 30% to 60%, from 35% to 60%, from 40% to 60%, from 10% to 40%, from 20% to 40%, from 30% to 40%, from 30% to 50%, or from 10% to 50%. In one embodiment, the amount of crosspolymer is around at least 5%. In an aspect of this embodiment, the amount of crosspolymer is at least 5%, at least 10%, at least 20%, at least 30%, or at least 40%.

The crosspolymer can also be replaced by plant oleosomes (oilbodies) as natural substitute. The plant olesomes include, without limitation, oleosomes from almond (Prunus dulcis); anise (Pimpinella anisum); avocado (Persea spp.); beach nut (Fagus sylvatica); borage (βoragio officinalis); Brazil nut (Bertholletia excelsa); candle nut (Aleuritis tiglium); carapa (Carapa guineensis); cashew nut (Ancardium occidentale); castor (Ricinus communis); coconut (Cocus nucifera); coriander (Coriandrum sativum); cottonseed (Gossypium spp.); crambe (Crambe abyssinica); Crepis alpina; croton (Croton tiglium); cucumber (Cucumis sativus); Cuphea spp.; dill (Anethum gravealis); Euphorbia lagascae; evening primrose (Oenothera biennis); Dimorphoteca pluvialis; false flax (Camolina sativa); fennel (Foeniculum vulgaris); groundnut (Arachis hypogaea); hazelnut (coryllus avellana); hemp (Cannabis sativa); honesty plant (Lunnaria annua); jojoba (Simmondsia chinensis); kapok fruit (Ceiba pentandra); kukui nut (Aleuritis moluccana); Lesquerella spp., linseed/flax (Linum usitatissimum); lupin (Lupinus spp.); macademia nut (Macademia spp.); maize (Zea mays); meadow foam (Limnanthes alba); mustard (βrassica spp. and Sinapis alba); olive (Olea spp.); oil palm (Elaeis guineeis); oiticia (Licania rigida); paw paw (Assimina triloba); pecan (Juglandaceae spp.); perilla (Perilla frutescens); physic nut (Gatropha curcas); pilinut (Canarium ovatum); pine nut (pine spp.); pistachio (Pistachio vera); pongam (Bongamin glabra); poppy seed (Papaver soniferum); pumpkin (Cucurbita pepo); rapeseed (Brassica spp.); safflower (Carthamus tinctorius); sesame seed (Sesamum indicum); soybean (Glycine max); squash (Cucurbita maxima); sal tree (Shorea rubusha); Stokes aster (Stokesia laevis); sunflower (Helianthus annuus); tukuma (Astocarya spp.); rung nut (Aleuritis cordata); vemonia (Vernonia galamensis); and mixtures thereof. In one embodiment, the oleosomes are prepared from plant seeds from the group of plant species comprising: rapeseed (Brassica spp.), soybean (Glycine max), sunflower (Helianthus annuus), oil palm (Elaeis guineeis), cottonseed (Gossypium spp.), groundnut (Arachis hypogaea), coconut (Cocus nucifera), castor (Ricinus communis), safflower (Carthamus tinctorius), mustard (Brassica spp. and Sinapis alba), coriander (Coriandrum sativum), squash (Cucurbita maxima), linseed/flax (Linum usitatissimum), Brazil nut (Bertholletia excelsa), jojoba (Simmondsia chinensis), maize (Zea mays), crambe (Crambe abyssinica) and eruca (Eruca sativa). In one embodiment, oleosomes prepared from safflower (Carthamus tinctorius) are used.

The present compositions also optionally include a medium to high melting lipid (esters, fatty acids and their derivatives, including waxes made from esters of long chain fatty acids). In one embodiment, the medium to high melting lipid has a melting point ranging from about 30° C. to about 80° C. In an aspect of this embodiment, the high melting lipid and wax has a melting point of at least 30° C., at least 40° C., at least 50° C., at least 60° C., at least 65° C., at least 70° C., at least 75° C., or at least 80° C.

In one embodiment, the medium to high melting lipid is selected from the one or more of plant butters: shea butter, apricot butter, cocoa butter, mango butter, kokum butter, illipe butter, cupuacu butter, murumuru seed butter, acrocomia aculeata butter, astrocaryum tucuma butter, attalea maripa butter, bassia latifolia seed butter, camellia reticulate seed butter, carpotroche brasiliensis seed butter, coconut seed butter, dipteryx odorate seed butter, palm butter, strawberry seed butter, sunflower seed butter, grcinia indica seed butter, walnut seed butter, irvingia gabonensis kernel butter, sesame seed butter, avocado butter, pentadesma butyracea seed butter, jojoba butter, shorea robusta seed butter, Theobroma grandiflorum seed butter, trichilia seed butter, virola surinamensis seed butter, pouteria sapota seed butter, butanoic acid (butyric acid), hexanoic acid (caproic acid), octanoic acid (acrylic acid), decanoic acid (capric acid), dodecanoic acid (lauric acid), tetradecanoic acid (myristic acid) hexadecanoic acid (palmitic acid), octadecanoic acid (stearic acid), tetracosanoic acid (lignoceric acid), eicosanoic acid (arachidic acid), and tribehenin.

In one embodiment, hydrogenated plant oil may be substituted for the high melting point lipid. Although the melting point of the hydrogenated plant oil is lower, if the composition is a gel or not a crème, it may be useful, especially to obtain a serum-like consistency.

In one embodiment, medium to high melting lipid is a wax made from esters of long chain fatty acids. In one embodiment, the ester wax of long chain fatty acids include, without limitation, a wax from following group: plant waxes: Rhus Succedanea Fruit Wax, Polianthes Tuberosa Flower Wax, Helianthus Annuus (Sunflower) Seed Wax, Rosa Multiflora Flower Wax, Citrus Aurantium Dulcis (Orange) Flower Wax, Narcissus Poeticus Flower Wax, Acacia Decurrens Flower Wax, Jasminum Grandiflorum (Jasmine) Flower Wax, Nelumbo Nucifera Flower Wax, Candelilla Cera, Cistus Ladaniferus Flower Wax, Citrus Aurantium Amara (Bitter Orange), Cananga Odorata Flower Wax, Citrus Limon (Lemon) Peel Wax, Copemicia Cerifera (Camauba) Wax, Copemicia Cerifera Cera, Euphorbia Cerifera (Candelilla) Wax, Cytisus Scoparius Flower Wax, Myrica Cerifera (Bayberry) Fruit Wax, Tuberose Wax, Palm Kernel Wax, Helichrysum Angustifolium Wax, Tribehenin and Ericerus Pela Wax. Animal derived waxes of interest in the present compositions include, without limitation: Beeswax, Lanolin Wax or Petrolatum wax.

In one embodiment, the amount of medium to high melting lipid is about 1% to 50% by weight. In one embodiment, the amount of high melting point lipid ranges from about 1% to 50%, 1% to 40%, 1% to 35%, 1% to 30%, 1% to 25%, 1% to 20%, 1% to 15%, 1% to 10%, 1% to 9%, 1% to 8%, 1% to 7%, 1% to 6%, 1% to 5%, 1% to 4%, 1% to 3%, or 1% to 2%. In another embodiment, the amount of high melting point lipid is at least about 5%, at least 6%, at least 7%, at least 8%, at least 9%, at least 10%, at least 11%, at least 12%, at least 13%, at least 14%, or at least 15%. In an embodiment, the amount of high melting point lipid is at most 50%, at most 40%, at most 35%, at most 30%, at most 25%, at most 20%, at most 15%, at most 10%, or at most 5%.

The present composition may optionally include a low melting point lipid. In one embodiment, the low melting point lipid has a melting point ranging from about 0° C. to 50°. In one embodiment, the melting point of the low melting point lipid is at most about 20° C., at most 15° C., at most 10° C., at most 9° C., at most 8° C., at most 7° C., at most 6° C., at most 5° C., or at most 4° C. In one embodiment, the low melting point lipid has a melting point ranging from about 0° C. to about 20° C., about 4° C. to about 17° C., about 4° C. to 18° C., about 4° C. to 19° C., about 4° C. to 20° C., about 4° C. to 25° C., about 4° C. to 30° C., about 4° C. to 35° C., about 4° C. to 40° C., about 4° C. to 45° C. or about 4° C. to 50° C.

Low melting point lipid oils of interest include, without limitation, an oil selected from one or more of the group consisting of cyclopentasiloxane, olus oil, camelina sativa oil, vegetable oil, palm oil, camelina sativa seed oil, Abyssinian oil, apricot kernel oil, fractionated coconut oil, jojoba oil, mongongo nut oil, pine nut oil, cashew oil, beech nut oil, sweet almond oil, walnut oil, wheat germ oil, avocado oil, borage seed oil, cranberry seed oil, evening primrose oil, grape seed oil, hazelnut oil, kukui nut oil, macademia nut oil, meadowfoam oil, olive oil, peanut oil, pecan oil, pomegranate seed oil, rose hip oil, seabuckthorn berry oil, sesame seed oil, sunflower oil, watermelon seed oil, canola oil, camellia oil, tigernut oil, thistle oil, safflower oil, oat oil, marula oil, Moroccan oil, dika oil, fish oil, kapok seed oil, peanut oil, seje oil, sacha inchi oil, palm kemel oil, kenaf seed oil, and argan oil.

In one embodiment, the amount of low melting point lipid is about 1% to 50% by weight. In one embodiment, the amount of low melting point lipid ranges from about 1% to 50%, 1% to 40%, 1% to 35%, 1% to 30%, 1% to 25%, 1% to 20%, 1% to 15%, 1% to 10%, 1% to 9%, 1% to 8%, 1% to 7%, 1% to 6%, 1% to 5%, 1% to 4%, 1% to 3%, or 1% to 2%. In another embodiment, the amount of low melting point lipid is at least about 5%, at least 6%, at least 7%, at least 8%, at least 9%, at least 10%, at least 11%, at least 12%, at least 13%, at least 14%, or at least 15%. In an embodiment, the amount of low melting point lipid is at most 50%, at most 40%, at most 35%, at most 30%, at most 25%, at most 20%, at most 15%, at most 10%, or at most 5%.

The present compositions also include one or more volatile compound(s). Volatile compounds of interest include, without limitation, a silicon-containing volatile oil or a hydrocarbon for example, cyclomethicone, isododecane, undecane, tridecane, coconut alkanes, dimethylsiloxane, cyclopentasiloxane, polymethylphenylsilxoxane, decamethylcyclopentasiloxane.

The amount of volatile compound(s) may vary, but generally ranges from about 1% to 60% by weight. In one embodiment, the amount of volatile compound(s) is around at most 60%, at most 50%, at most 40%, at most 30%, at most 25%, at most 20%, at most 15%, or at most 10%. In one embodiment, the amount of volatile compound(s) ranges from 5% to 60%, from 10% to 60%, from 20% to 60%, from 25% to 60%, from 30% to 60%, from 35% to 60%, from 40% to 60%, from 10% to 40%, from 20% to 40%, from 30% to 40%, from 30% to 50%, or from 10% to 50%. In one embodiment, the amount of volatile compound(s) is around at least 5%. In an aspect of this embodiment, the amount of volatile compound(s) is at least 5%, at least 10%, at least 20%, at least 30%, or at least 40%.

The compositions described herein are substantially anhydrous. “Substantially Anhydrous” (used synonymously with “non-aqueous”) in accordance with the present invention means that the formulation, be it a lotion, gel, crème, cosmetic base or cosmetic composition, exists substantially in the absence of water. While formulations which are completely water free or which have only trace amounts of water are most desired, the term is meant to encompass total water contents of up to about 5% by weight. In one embodiment, the claimed compositions contain less than about 4% by weight water. In a further embodiment, the present compositions contain less than about 3%, less than about 2%, less than about 1%, or less than about 0.5% water by weight. In one embodiment, the present compositions contain up to about 0.5%, up to about 1%, up to about 2%, up to about 3%, up to about 4%, or up to about 5% water by weight.

As defined herein, stability is tested by placing the composition in a controlled environment chamber for 8 weeks at 25° C. In this test, the physical condition of the sample is inspected as it is placed in the chamber. The sample is then inspected again at 24 hours, 3 days, 1 week, 2 weeks, 4 weeks and 8 weeks. At each inspection, the sample is examined for abnormalities in the composition such as phase separation if the composition is in the form of an emulsion, bending or leaning if the composition is in stick form, melting, or syneresis (or sweating). The stability is further tested by repeating the 8-week test at 40° C., 45° C., 50° C. and under freeze-thaw conditions. A composition is considered to lack stability if in any of these tests an abnormality that impedes functioning of the composition is observed. The skilled artisan will readily recognize an abnormality that impedes functioning of a composition based on the intended application.

The composition of the present invention may be in any form. For example, it may be a paste, a solid, a serum, a gel, or a cream. It may be an emulsion, or a solid, rigid or supple gel, including anhydrous gels. The composition may be in the form of a lotion. The composition can also be in a form chosen from a translucent anhydrous gel and a transparent anhydrous gel. The composition of the invention may, for example, comprise an external or continuous fatty phase.

The composition may be anhydrous. In another embodiment, the composition of the invention may be transparent or clear, including for example, a composition without pigments. The composition can also be a molded composition or cast as a stick or a dish. The composition in one embodiment is a solid such as a molded stick or a poured stick. The compositions of the present invention may also be in the form a lip composition such as a lipstick or a liquid lip color or a foundation.

The composition may also be an aerosolized liquid, serum, gel, foam or crème. Methods of aerosolization and foams are recognized in the art.

The formulations of the present invention, as a base system for the addition of an active ingredient, a lotion, or a cosmetic base and cosmetic composition, can also be described by certain advantageous rheological properties.

Viscosity: The complex modulus or G*(ω) can be measured by dynamic oscillation. The ratio of storage modulus or elastic modulus (G′(ω)) divided by loss modulus (G″(ω)) is a measure of same. These can be measured at various frequencies. However, for use herein, measurement can be taken at either 0.1 rad/s or 100 rad/s or both. Complex modulus can be measured using a controlled-stress rheometer Model AR2000 (TA instrument) with a parallel plate geometry of a diameter of 40 millimeters. The gap is set at 1 millimeter. The rheological measurements are performed at 25° C. The measurement of dynamic oscillation is performed in a linear viscoelastic regime at an applied stress of 1.0 Pascal. From this experiment, the storage modulus and the loss modulus are obtained as a function of frequency from (.phi.)=100 rad/s to 0.1 rad/s.

The real or in-phase component of the complex dynamic viscosity η′(ω) is related to the loss modulus G″(ω) by the formula η′(ω)=G″(ω))/ω. See Viscoelastic Properties of Polymers, John D. Ferry, John Wiley & Sons, third edition, 1980, or Chapter 6, page 242 in Polymer Viscoelasticity:Stress and Strain in Practice, Evaristo Riande et al, Marcel Decker, Inc., 2000.

When frequency is 100 rad/sec, the relationship G′/G″ can range from about 1 to about 100. In another embodiment, it can range from about 1.2 to about 50, and in another embodiment, from about 1.3 to about 25. In another embodiment, it can range from about 1.5 to about 7. When measured at a frequency of 0.1 rad/sec, the ratios are about 0.1 to about 100, about 1 to about 50, about 2.5 to about 25, and about 3 to about 4, respectively. In a preferred embodiment, the formulations of the invention will meet both ranges at 100 and 0.1 rad/s as described earlier herein.

Dynamic viscosity ranges in accordance with one embodiment of the present invention can range from about 1×10¹ to about 9×10⁶ Pas. In another embodiment, dynamic viscosity can range from about 1×10² to about 5×10⁶ Pas. In yet another embodiment from about 1×10³ to about 1×10⁶ Pas. In still another embodiment, dynamic viscosity can range from about 2×10³ to about 1×10⁵ Pas.

In another embodiment, G′/G″ at 100 rad/s is from about 1 to about 100, G′/G″ at 0.1 rad/s is from about 0.1 to about 100 and dynamic viscosity is about 1×10¹ to about 9×10⁶ Pas. In another embodiment, G′/G″ at 100 rad/s is from about 1.2 to about 50, G′/G″ at 0.1 rad/s is from about 1 to about 50 and dynamic viscosity is about 1×10² to about 5×10⁶ Pas. In yet another embodiment, G′/G″ at 100 rad/s is from about 1.3 to about 25, G′/G″ at 0.1 rad/s is from about 2.5 to about 25 and dynamic viscosity is about 1×10³ to about 1×10⁶ Pas. In still another embodiment, G′/G″ at 100 rad/s is from about 1.5 to about 7, G′/G″ at 0.1 rad/s is from about 3 to about 4 and dynamic viscosity is about 2×10³ to about 1×10⁵ Pas. It is also preferred that these formulations also have the hardness discussed elsewhere herein.

Additional ingredients as described herein may include, without limitation, therapeutically active ingredients, pigments, colorant, fragrances or other cosmetic additives. In one embodiment, the composition contains more than one additional active ingredient. In another embodiment, the composition contains no additional active ingredients.

Therapeutically active ingredients as used herein include, without limitation, a chemical substance or organism which when applied topically may prevent, reduce, or alleviate physical symptoms of a condition or disease. Conditions or diseases may include, but are not limited to, skin wrinkle prevention or alleviation, decreasing sweat production, decreasing or alleviating body odor, skin lightening (alleviating hyperpigmentation), increasing collagen production or retention, moisturizing skin, prevention or alleviation of hair loss, improving hair growth, treating or alleviating acne, wound healing, scar prevention or alleviation, preventing or alleviating sun damage.

Examples of therapeutically active ingredients of interest include steviocide (anti-inflammation), vitamin E, retinol and vitamin A. For decreasing body odor or acne, an antibiotic or biocide-might be used. Biocide or antibiotic of interest include one or more of benzyl peroxide, salicylic acid, a sulfonamide, an aminoglycoside, a quinolone, a oxazoladinone, gramicidin, or a tyrocidine. For decreasing sweat production, an anti-perspirant might be used. Examples of antiperspirants include, but are not limited to: aluminum chlorohydrate, aluminum zirconium tetrachlorohydrex gly, aluminum sulfate, ferric chloride hexahydrate, zirconium, and zirconyl chloride octahydrate. A sunscreen might be added to prevent sun damage to the skin or hair. Sunscreens include, without limitation, zinc oxide, titanium dioxide, oxybenzone, 3-(4-methylbenzylidene)-camphor (4-MBC), octyl-methoxycinnamate (OMC), octyl-dimethyl-PABA (OD-PABA), bexophenome-3 (Bp-3) and homosalate. For preventing/alleviating hair loss and/or increasing hair growth, minoxidil and/or phytosteros might be used.

The following biocides may be used as active ingredients for the prevention/alleviation of acne, wound healing, and prevention/alleviation of body odor: tea tree oil, benzyl peroxide, retinoic acid, glycolic acid, phytosterols, sialic acid, clindamycin hydrochloride, resorcinol, salicylic acid, or tretinoin powder might be included. Biocides include benzalkonium chloride, Benzyldimethylhexadecylammonium Chloride, Benzyldimethyltetradecylammonium Chloride, boric acid, cetylpyridinium chloride, cetyltrimethylammonium bromide, cetyltrimethylammonium p-Touenesulfonate, chloramine-T trihydrate, chlorhexidine diacetate, chlorhexidine dihydrochloride, chlorophene, hcloroxylenol, ciclopirox olamine, clotrimazole, coal tar, (+)-alpha-pinene, 2,4-Dichloro-3,5-dimethylphenol, dodecyltimethylammonium bromide, domiphen bromide, ethylhexadecyldimethylammonium broimide, glutaric dialdehyde, hexachlorophene, hexadecyltrimethylammonium chloride, 4-hexaresorcinol, hydrogen peroxide, 8-quinolinol, ichtammol, iodoform, (−)-alpha-pinene, mercuric oxide (yellow or red), methenamine, methylbenzenthonium chloride, p-chlorophenol, phenol, 2-phenylphenol, phenyl salicylate, povidone-iodine, proprionic acid, resorcinol, silver oxide, sodium bromide, sodium o-phenylphenate, sodium phenoxide, tetradecyltimethylammonium bromide, tetramethylthiuram, thiabendazole, triacetin, triclocarbon, triclosan, tris(hydroxymethyl)nitromethane, undecylenic acid, (=/−)-usnic acid, zinc aspartate complex, zinc borate, zinc chloride, zinc citrate, zinc di-n-butyldithiocarbamate, zinc gluconate, zinc phenylsulfonate, zinc pyrithione, zinc sulfate, zinc undecylenate, and dequalinium chloride.

In addition, for wound healing or scar prevention/alleviation hyaluronic acid or a derivative thereof, sialic acid, glycolic acid, retinoic acid, and/or urea might be added.

Probiotic extract and fermentation filtrate may also be used as active ingredients, of interest include powdered yeast such as yeasts, Saccharomyces Lysate Extract powder, Lactbacillus Ferment Lysate filtrate, or yogurt extract.

Stem cells may also be used as active ingredients. Stem cells are formulated in this substantially anhydrous system with extracts of botanical stem cells or mammalian stem cells.

The amount of the additional active ingredient varies by the active ingredient and the purpose for which it is being used. Moreover, more than one additional active ingredient may be included. Generally, the amount of a single additional active ingredient is about at most 10%, at most 9%, at most 8%, at most 7%, at most 6%, at most 5% at most 4%, at most 3%, at most 2%, or at most 1%. In one aspect, the amount of a single additional active ingredient ranges from about 0.01% by weight to 10% by weight. In one embodiment, the amount of a single additional active ingredient ranges from about 0.01% to 10% by weight, 0.05% to 10% by weight, 0.1% to 10% by weight, 1% to 10% by weight, 2% to 10% by weight, 3% to 10% by weight, 4% to 10% by weight 5% to 10% by weight, and 6% to 10% by weight. Examples of ingredients having such ranges include, but are not limited to, the sunscreens (such as zinc oxide and titanium dioxide), some antibiotics (such as benzyl peroxide), and others. In another aspect the amount of additional active ingredient ranges from about 0.01% to 5%, about 0.05% to 5% about 0.1% to 5%, about 0.5% to 5%, about 1% to 5%, about 2% to 5%, or about 3% to about 5%. Examples of additional active ingredients having such ranges include, but are not limited to phytosterols, minoxidil, vitamin A, vitamin E, retinyl palmitate, and others.

In one embodiment, the amount of all additional ingredients (one or more) is at most about 20% of the total composition. In an aspect the amount of all additional ingredients is at most 20%, at most 19%, at most 18%, at most 17%, at most 16%, at most 15%, at most 14% at most 13%, at most 12%, at most 11% at most 10%, at most 9%, at most 8%, at most 8%, at most 7%, at most 6%, at most 5%, at most 4%, at most 3%, at most 2%, or at most 1%. In one embodiment, the composition contains no additional active ingredients.

Pigments, Natural Colorant and Fragrances: The present compositions may include one or both of pigments, natural colorants, or fragrances. The amount of pigment or fragrance may vary depending on use, but when present is generally within the range from about 0.01% by weight to 5% by weight. In one embodiment, the amount of fragrance ranges from about 0.01% by weight to 1% by weight, 0.05% by weight to 1% by weight, 0.1% by weight to 1% by weight, 0.2% by weight to 1% by weight, 0.3% by weight to 1% by weight, 0.4% by weight to 1% by weight, 0.5% by weight to 1% by weight, 0.01% by weight to 0.75% by weight, 0.05% by weight to 0.75% by weight, 0.1% by weight to 0.75% by weight, 0.2% by weight to 0.75% by weight, 0.01% by weight to 0.5% by weight 0.05% by weight to 0.5% by weight, 0.1% by weight to 0.5% by weight, 0.2% by weight to 0.5% by weight, or 0.3% by weight to 0.5% by weight.

Pigments suitable for use herein may be inorganic and organic colors/pigments suitable for use in makeup compositions. These are usually aluminum, barium or calcium salts or lakes. Lakes are either a pigment that is extended or reduced with a solid diluent or an organic pigment that is prepared by the precipitation of a water-soluble dye on an adsorptive surface, which usually is aluminum hydrate. A lake also forms from precipitation of an insoluble salt from an acid or basic dye. Calcium and barium lakes are also used herein.

Preferred lakes of the present invention are Red 3 Aluminum Lake, Red 21 Aluminum Lake, Red 27 Aluminum Lake, Red 28 Aluminum Lake, Red 33 Aluminum Lake, Yellow 5 Aluminum Lake, Yellow 6 Aluminum Lake, Yellow 10 Aluminum Lake, Orange 5 Aluminum Lake and Blue 1 Aluminum Lake, Red 6 Barium Lake, Red 7 Calcium Lake, Red 30 Talc Lake, and Red 30 Aluminum Lake.

Other colors and pigments can also be included in the present compositions, such as dyes and pearls, titanium oxides, Red 6, Red 21, Blue 1, Orange 5, and Green 5 dyes, chalk, talc, iron oxides and titanated micas.

Natural colorants of interest include without limitation, Curcuma Longa (Turmeric) Root Extract, Melia Azadirachta Leaf Extract, Melia Azadirachta Flower Extract, Melia Azadirachta Bark Extract, Pearl Powder, Moringa Pterygosperma Seed Oil, Ocimum Sanctum Extract, Ocimum Basilicum (Basil) Extract, Solanum Melongena (Eggplant) Fruit Extract, Moringa Extract, Moringa Pterygosperma Seed Oil, Eclipta Alba Hassk, Eclipta Prostrata Extract, Amber, Coccinia Indica Leaf Extract, Coccinia Indica Extract, Aloe Barbadensis Flower Extract, Aloe Barbadensis Flower Extract, Eclipta Prostrata Extract, Corallina Officinalis Extract, Eclipta Prostrata Extract, and Corallina Officinalis Extract.

Compositions of the present invention contain sufficient pigments or colorants to provide the look sought by the user.

Additional cosmetic ingredients may include, without limitation, bulking agents, binders, and texturing agents. A filler as described herein may be amorphous silica compounds (e.g., silica dimethyl silylate), aluminum silicate, activated alumina, ammonium carbonate, powdered bentonite, calcium carbonate, calcium phosphate, calcium caseinate, calcium silicate, calcium sulfate, microcrystalline cellulose, chitin, corn starch, corn gluten meal, dextran, dextrin, diatomaceous earth, hectorite, kaolin, magnesium carbonate, magnesium phosphate, magnesium silicate, magnesium stearate, magnesium sulfate, magnesium trisilicate, silica gel, soybean flour, potato starch, rice starch, wheat starch, and talc.

Binders of interest include acacia powder, agar powder, alginic acid, amylopectin, behenyl alcohol, benzoin gum, calcium caseinate, carboxymethylcellulose sodium, ceresin wax, collodion, corn starch, croscarmellose sodium, damar gum, dextran, dextrin, gelatin, ethylcellulose, gluten, glycerin, guar gum, hydroxyethyl cellulose, hydroxypropyl cellulose, hypromellose, isopropyl myristate, karaya gum, locust bean gum, maltodextrin, mannitol, mastic gum, methylcellulose, mannitol, multiwax, n-Octyltriethyoxysilane, VP/VA copolymer, n-Octyltrimethoxysilane, pectin, polyacrylamide, poly(styrene0comaleic anhydride), polyvinyl acetate, polyvinyl alcohol, polyvinyl butyral, povidone, pullulan, polyvinylpyrrolidone, rosin gum, shellac gum, sodium alginate, sodium starch glycolate, sorbitol, potato starch, wheat starch, stearyl palmitate, beeswax (and substitutes thereof), tragacanth powder, tridecafuoro-1,1,2,2,-tetrahydrooctyl-1-triethoxysilane, 3-(trimethoxysilyl)propyl Methacrylate, white wax, yellow wax beaded beeswax, xanthan gum, and poly(vinyl methyl Ether).

Texturizing agent of interest include chemical slip modifiers and conditioning agents. Chemical slip modifiers include, without limitation, caprylyl dimethicone, polymethylsilsesquioxane, aluminum silicate, boron nitride, microcrystalline cellulose, kaolin powder, magnesium aluminum silicate, magnesium silicate, magnesium trisilicate, oleamide, polytetrafluoroethylene, talc powder, veegum, zinc laurate, zinc myristate, zinc palmitate, zinc resinate, and zinc stearate.

The amount of bulking agent, binder, or texturing agent is generally less than about 25%. In one embodiment, the amount of bulking agent is at most 25%, at most 24%, at most 23%, at most 22%, at most 21%, at most 20%, at most 19%, at most 18%, at most 17%, at most 16%, at most 15%, at most 14%, at most 13%, at most 12%, at most 11%, at most 10%, at most 9%, at most 8%, at most 7%, at most 6%, at most 5%, at most 4%, at most 3%, at most 2%, or at most 1%.

Although the present invention has been described in considerable detail with reference to certain preferred versions thereof, other versions are possible. For example, combinations of each of the ingredients may be employed.

The following examples serve to illustrate the invention. These examples are in no way intended to limit the scope of the present compositions of methods.

### EXAMPLES

The following non-limiting examples are provided for illustrative purposes only in order to facilitate a more complete understanding of the disclosed subject matter. These examples should not be construed to limit any of the embodiments described in the present specification, including those pertaining to the apparatus used therein and the methods of using the present apparatus.

Sources of Materials:

Materials may be obtained from multiple sources, and may be substituted with other ingredients having the same INCI Name. However, ingredients may be obtained or used from the following sources, without limitation:

TABLE 1
Potential Sources of Representative Materials

| INCI Name | Source | Product Name |
|---|---|---|
| Ascorbic Acid | Specialty Ingredient | Vita C Powder |
| | Technology | |
| Tetrahexyldecyl Ascorbate | Barnet | BV-OSC |
| Dimethicone | Siltech | SILTECH F-350 |
| Dimethicone Crosspolymer | Specialty Ingredient | Dimethicone |
| | Technology | Crosspolymer |
| Pentaerythrityl Tetraisostearate | Croda | CRODAMOL PTIS |
| Dimethicone/Vinyl Dimethicone | Dow Corning | DC 9701 |
| Crosspolymer (and) Silica | | |
| Cyclopentasiloxane | Botanigenics | Botanisil CP-33 |
| Tangerine (Citrus Tangerina) Oil | Citrus & Allied Ess. | OIL TANGERINE |
| | | DANCY CP FCC |
| Fragrance | Fragrance | CITRUS LIGHT |
| SAFFLOWER OLEOSOMES | Specialty Ingredient | Carthamus Tinctorius |
| | Technology | (Safflower) Oleosomes |
| Hyaluronic Filling Spheres | BASF | Sodium Hyaluronate |
| Olus Oil (and) Camelina Sativa Oil | AAK | Lipex ® Omega 3/6 |
| [EU] - Vegetable Oil (and) | | |
| Camelina Sativa Seed Oil [US] | | |
| Crambe Abyssinica Seed Oil | Premier | ABYSSINIAN OIL |
| Corn (Zea Mays) Oil [and] Glyceryl | Eastman | TENOX 6 |
| Oleate [and] Propylene Glycol | | |
| [and] BHA [and] | | |
| Carthamus Tinctorius (Safflower) | Active Organics | Actiphyte of Chamomile |
| Seed Oil | | Lipo S |
| jojoba oil | Lipo Chemicals | Lipovol J |
| coconut oil | Integrity Ingredient | coconut oil |
| vitamin A palmitate | Retinyl Palmitate | Roche |
| Hydroxypropyl Beta Cyclodextrine | Lipo Chemicals | LIPO CD-SA |
| [and] Salicylic Acid | | |
| tocopherol | DSM | Tocopherol |
| tocopherol acetate | DSM | vitamin E acetate |
| Retinyl Palmitate | DSM | Vitamin A palmitate |
| | | P1.0 |
| sodium hyaluronate | Specialty Ingredient | hyaluronic acid LMW |
| | Technology | |
| PPG-2 Myristyl Ether Propinonate | Croda | CRODAMOL PMP |
| Stearyl Alcohol | Lipo Chemicals | LIPOCOL S |
| Cetearyl Alcohol [and] | Croda | INCROQUAT |
| Behenyltrimonium Chloride | | BEHENYL TMC 25 |
| Cetearyl Alcohol [and] Polysorbate | Arch Chemicals | BROOKS WAX P |
| 60 | | |
| Crambe Abyssinica Seed Oil | Premier | ABYSSINIAN OIL |
| Polymethylsilsesquioxane, | Active Concepts | SilDerm Emulsifying CS |
| Cyclopentasiloxane | | |
| Butyrospermum Parkii (Shea) | AAK | SHEA BUTTER |
| Butter | | |
| apricot butter | Ross Oganics | Citrus Tangerina |
| | | (Tangerine) Peel Oil |
| beeswax | Strahl & Pitsch | beeswax |
| apricot butter | Integrity Ingredient | Citrus Tangerina |
| | | (Tangerine) Peel Oil |
| Glycerin | Univar | GLYCERIN |

### Comparative Example 1

### Conventional Water-in-Oil Emulsion

A composition having the following ingredients was generated:

TABLE 2
Comparative Example 1

| INGREDIENT | | | |
|---|---|---|---|
| NAME | Mfr./Vendor | US/INCI (EU/INCI) Name | % w/w |
| “A” | | | |
| INCROQUAT | Croda | Cetearyl Alcohol [and] Behenyltrimonium | 3.50000 |
| BEHENYL TMC | | Chloride | |
| 25 | | | |
| BROOKS WAX P | Arch | Cetearyl Alcohol [and] Polysorbate 60 | 3.00000 |
| | Chemicals | | |
| LIPOCOL S | Lipo | Stearyl Alcohol | 1.00000 |
| | Chemicals | | |
| PERMETHYL 99 | Presperse | Isododecane | 2.00000 |
| A | | | |
| PERMETHYL 101 | Presperse | Isohexadecane | 3.00000 |
| A | | | |
| CRODAMOL PMP | Croda | PPG-2 Myristyl Ether Propinonate | 3.00000 |
| CRODAMOL PTIS | Croda | Pentaerythrityl Tetraisostearate | 2.00000 |
| SILTECH F-350 | Siltech | Dimethicone | 0.50000 |
| TOCOPHEROL | DSM | Tocopherol | 0.15000 |
| TENOX 6 | Eastman | Corn (Zea Mays) Oil [and] Glyceryl Oleate | |
| | | [and] Propylene Glycol [and] BHA [and] | |
| | | BHT [and] Propyl Gallate [and] Citric Acid | 0.25000 |
| VITAMIN A | Roche | Retinyl Palmitate | 0.05000 |
| PALMITATE | | | |
| “B” | | | 0.25000 |
| DI WATER | | Water (Aqua) | 74.58000 |
| VERSENE 100 | Dow | Tetrasodium EDTA | 0.05000 |
| | Chemicals | | |
| VANTOX SBS | UPI | Sodium Bisulflte | 0.02000 |
| “C” | | | |
| PHENONIP | Nipa/Wilchem | Phenoxyethanol [and] Methylparaben [and] | |
| | | Butylparaben [and] Ethylparaben | |
| | | [and] Propylparaben | 1.00000 |
| LIPO CD-SA | Lipo | Hydroxypropyl Beta Cyclodextrine [and] | 0.30000 |
| | Chemicals | Salicylic Acid | |
| Vita C POWDER | Specialty | Ascobic Acid | 5.00000 |
| | Ingredient | | |
| | Technology | | |
| “D” | | | |
| OIL TANGERINE | Citrus & | Tangerine (Citrus Tangerina) Oil | 0.35000 |
| DANCY CP FCC | Allied Ess. | | |
| | | Total | 100.0000 |

Procedure:
1. The ingredients of group A are combined and heated to 75° C. and mixed until homogenous.
2. The ingredients of group B are combined and heated to 80° C. and mixed until homogenous.
3. When both mixture A and mixture B are homogenous, mixture B is slowly added to A and mixed thoroughly.
4. Once mixed, the combined mixture AB is cooled.
5. When the temperature of mixture AB reaches 50-55° C., the C ingredients are added to the mixture AB. They should be added in the order listed. For instance, it is useful to add the salicylic acid to the mixture AB before adding the ascorbic acid to acid to acidify the mixture and preserve the ascorbic acid. In addition, the decrease in temperature is useful to preserve the ascorbic acid and temperatures above 60° C. should preferably be avoided.
6. The mixture is allowed to cool further, until it reaches a temperature of 35-40° C.
7. Once at the 35-40° C. temperature, the ingredient(s) of D are added. If there are multiple ingredients of D, then they should be mixed well between each addition, and the composition should be further mixed while cooling.
8. Finally, the pH should be adjusted with Citric Acid to pH of 3.0-3.5, if necessary.

The mixture of Comparative Example 1 was prepared and then stored at 45° C. for three months to simulate degradation during long-term storage conditions at room temperature. For instance, the 45° C. for three months corresponds to 2 years shelf life at 25° C.

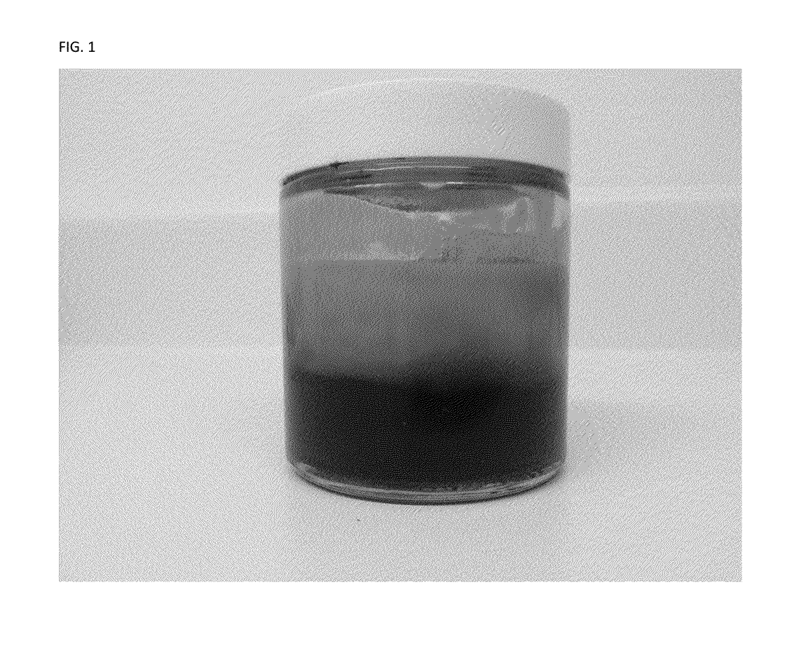

As shown by FIG. 1, the composition achieved after the simulated storage conditions is separated into multiple phases and brown or brownish yellow.

### Comparative Example 2

### 12% Vitamin C Serum with 5% Water

In addition, the amount of water can detrimentally affect the stability of the system of the present invention.

### 12% Vitamlne C Serum

TABLE 3
Comparative Example 2

| INGREDIENT NAME | Mfr/Vendor | INCI Name | % w/w |
|---|---|---|---|
| A | | | |
| PROPYLENE | Open | Propylene Glycol | 40.0000 |
| GLYCOL | | | |
| VITA C POWDER | Specialty | Ascorbic Acid | 12.0000 |
| | Ingredient | | |
| | Technology | | |
| GLYCERIN | Univar | Glycerin | 7.0000 |
| DEIONIZED WATER | | Water | 5.0000 |
| B | | | |
| SD ALCOHOL 40B | Mid West Grain | Ethyl Alcohol | 35.0000 |
| VITAMIN E | DSM | Tocopherol | 0.5000 |
| ACETATE | | Acetate | |
| CITRUS LIGHT | Fragrance west | Fragrance | 0.5000 |

| | |
|---|---|
| TOTAL | 100.0000 |

Procedure:

1—Weigh PROPLENE GLYCOL in to the mixing tank. Under high mixing, shift in ASCORBIC ACID.
2—Add in the rest of “A” ingredients in the order indicated and start to heat the batch to 55-60° C.
3—When all the solid is dissolved and a clear solution is formed, remove the batch from the heat and let the batch cool naturally. Make sure no solid crystal out.
4—In a separate vessel, combine “B” ingredients and mix until uniform.
5—When batch temperature reaches 40° C., add premixed “B” ingredients. Continue to mix until the mixture cools to room temperature.

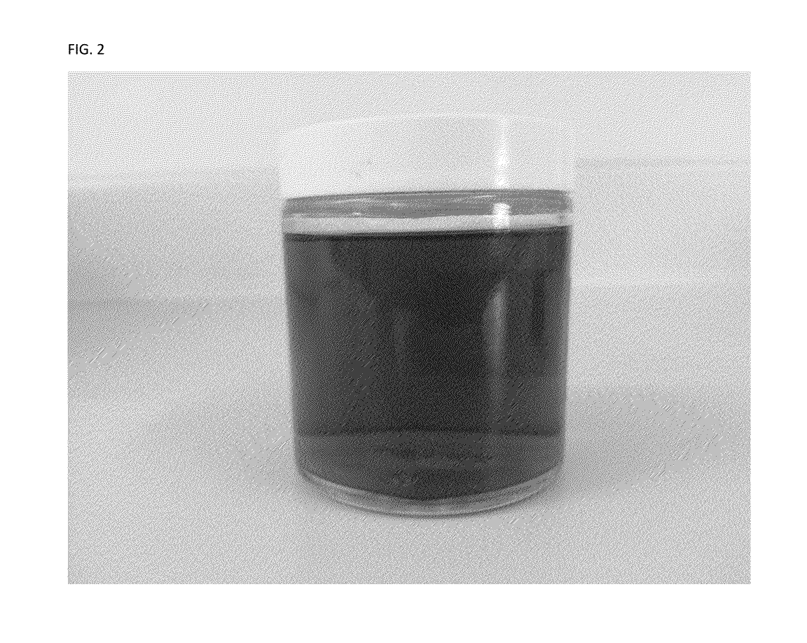

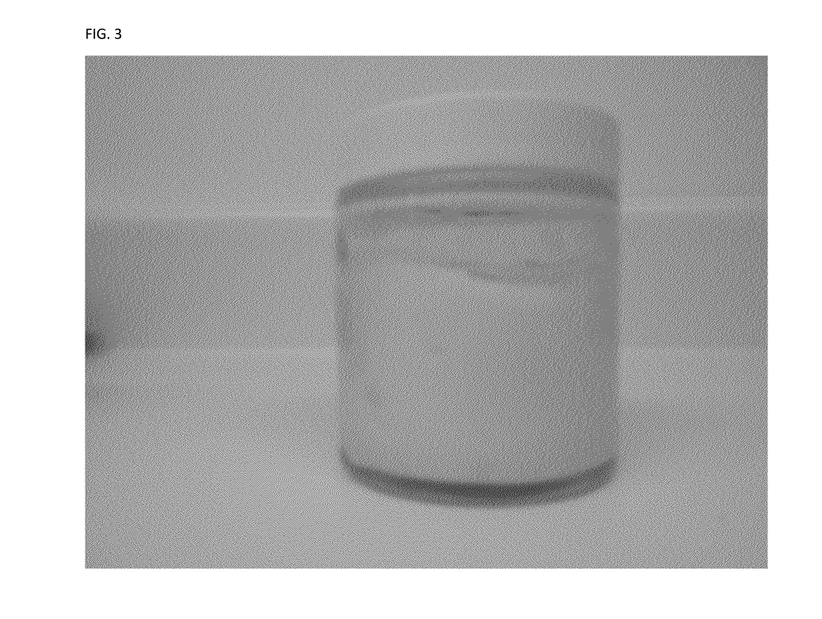

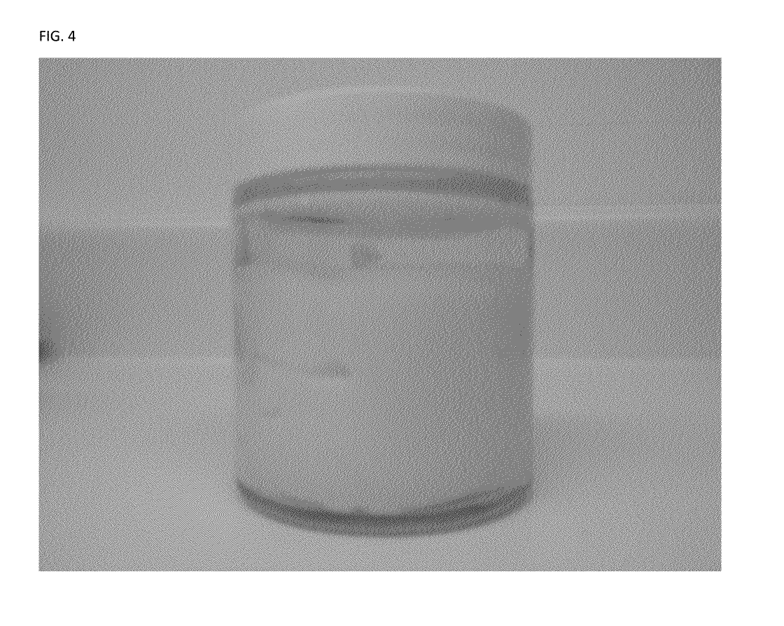

The serum upon cooling was clear with a light fragrance. The serum was placed in an incubator for three months at 45° C. to test its stability. The serum after three months is shown in FIG. 2. It is brown and not suitable for administration or sale.

### Example 1

### 10% Vitamin C Cream

A composition of having the ingredients of Table 4 was made as follows:

TABLE 4
Example 1

| INCI Name | % w/w |
|---|---|
| A | |
| Ascorbic Acid | 10.000 |
| Dimethicone/Vinyl Dimethicone | 11.000 |
| Crosspolymer (and) Silica | |
| Carthamus Tinctorius (Safflower) | 2.000 |
| Oleosomes | |
| Dimethicone Crosspolymer | 10.000 |
| Cyclopentasiloxane | 57.400 |
| B | |
| Olus Oil (and) Camelina Sativa Oil [EU] - | 0.300 |
| Vegetable Oil (and) Camelina Sativa | |
| Seed Oil [US] | |
| Crambe Abyssinica Seed Oil | 3.000 |
| Butyrospermum Parkii (Shea) Butter | 2.000 |
| C | |
| Sodium Hyaluronate | 2.000 |
| Ethyhexyl Palmitate | |
| Silica Dimethyl Silylate | |
| Tetrahexyldecyl Ascorbate | 2.000 |
| Retinyl Palmitate | 0.100 |
| Fragrance | 0.200 |
| TOTAL | 100.000 |

Procedure:

1. Combine “A” and mix under high shear until all Vitamin C powder has been encapsulated into the oleosomes and crosspolymers.
2. Combine “B” and mix to homogenous and free of lump.
3. Slowing add “B” to “A” with low shear mixing. Mix until all the liquid has been folded into the anhydrous gel
4. Add “C” in the order indicated and mix well between each addition.

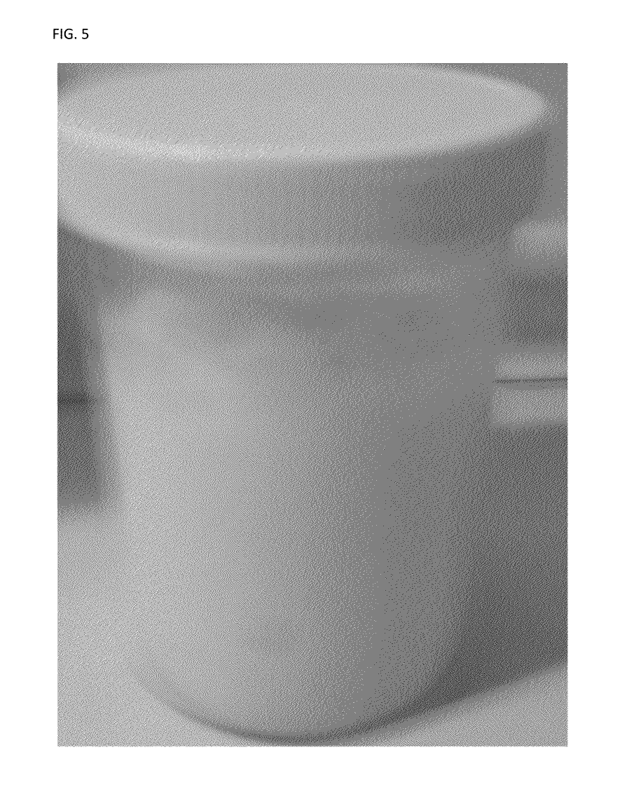

The resulting mixture had a soft texture (with no grittiness) and good glide. The cream shown in below picture was stable at 40° C. incubator for 3 months. Again a temperature of 40-45° C. for approximately 3 months is equivalent to a storage stability of 2 years at 25° C. FIG. 5 shows the cream immediately after mixing. FIG. 3 shows the cream made according to this embodiment after three months at 40° C.

### Example 2

### 10% Vitamin C Serum

TABLE 5
Example 2

| INCI Name | % w/w |
|---|---|
| A | |
| Ascorbic Acid | 10.000 |
| Dimethicone/Vinyl | 7.000 |
| Dimethicone Crosspolymer, | |
| Silica | |
| Carthamus Tinctorius | 12.000 |
| (Safflower) Oleosomes | |
| Dimethicone Crosspolymer | 5.000 |
| Cyclopentasiloxane | 44.500 |
| B | |
| Carthamus Tinctorius | 0.500 |
| (Safflower) Seed Oil | |
| Polymethylsilsesquioxane, | 10.000 |
| Cyclopentasiloxane | |
| Butyrospermum Parkii (Shea) | 1.000 |
| Butter | |
| Crambe Abyssinica Seed Oil | 2.000 |
| Citrus Tangerina (Tangerine) | 5.000 |
| Peel Oil | |
| C | |
| Sodium Hyaluronate | 2.000 |
| Ethyhexyl Palmitate | |
| Silica Dimethyl Silylate | |
| Retinyl Palmitate | 0.500 |
| Fragrance | 0.500 |
| Total: | 100.000 |

Ingredients were combined in a manner similar to that discussed in Example 1 above. The resulting mixture had a soft texture (with no grittiness), feels smooth and silky with good glide. The mixture stayed creamy white in an accelerated stability incubator of temperature 45° C. for 3 months. FIG. 5 shows the cream after the three month test.

The cream is softer and less viscous in texture when the amount of Dimethicone/Vinyl Dimethicone Crosspolymer and Dimethicone crosspolymer is decreased while the amount of Oleosome is increased.

### Example 3

### 12% Vitamin C Paste

TABLE 6
Example 3

| INCI Name | % w/w |
|---|---|
| Cyclopentasiloxane | 60.500 |
| Dimethicone Crosspolymer | 8.000 |
| Ascorbic Acid | 12.000 |
| Dimethicone/Vinyl | 3.000 |
| Dimethicone Crosspolymer, | |
| Silica | |
| Beeswax | 7.000 |
| Jojoba oil | 8.000 |
| Tocopherol acetate | 0.200 |
| Sodium Hyaluronate | 0.200 |
| Tetrahexyldecyl Ascorbate | 0.500 |
| Retinyl Palmitate | 0.300 |
| Fragrance | 0.300 |
| Total: | 100.00 |

Ingredients were combined in following procedure:

1. Combine “A” and mix under high shear until all Vitamin C powder has been encapsulated into the oleosomes and crosspolymers.

2. Combine “B” and heat to 65-75 C. Mix until free of particles. Then cool the mixture to 50-55° C.

3. Slowly add “A” to “B”. Mix until uniform.

4. Add “C” in the order indicated and mix well between each addition.

The resulting mixture had a harder and thick paste like texture (with no grittiness) and good glide. The mixture stayed creamy white in an accelerated stability incubator of temperature 45° C. for 3 months.

The cream became harder and had a more viscous in texture when beeswax was used to replace the butters.

### Example 4

### 14% Vitamin C Cream

TABLE 7
Example 4

| INCI Name | % w/w |
|---|---|
| Ascorbic Acid | 14.000 |
| Cyclopentasiloxane | 60.000 |
| Dimethicone Crosspolymer | 10.000 |
| Carthamus Tinctorius | 0.500 |
| (Safflower) Seed Oil | |
| Polymethylsilsesquioxane, | 6.200 |
| Cyclopentasiloxane | |
| Citrus Tangerina (Tangerine) | 10.000 |
| Peel Oil | |
| Coconut Oil | 1.000 |
| Retinyl Palmitate | 0.200 |
| Fragrance | 0.500 |
| Total: | 100.00 |

Ingredients were combined in a manner similar to that discussed in Example 1 above. The resulting mixture had a soft texture (with no grittiness), good glide, and feels silky and smooth. The mixture stayed creamy white in an accelerated stability incubator of 45° C. for 3 months.

A higher amount of Vitamin C level may give the mixture a more creamy look.

### Example 5

### 12% Ascorbic Acid

TABLE 8
Example 5

| Ingredient | % wt |
|---|---|
| Cyclomethicone (ie. Cyclopentasiloxane) | 40 |
| Ascorbic Acid | 12 |
| Dimethicone/Vinyl Dimethicone Crosspolymer | 10 |
| Dimethicone Crosspolymer | 20 |
| Silica Dimethyl Silylate | 3 |
| Oil (ie. Safflower oil) | 5 |
| Lipids (ie Shea Butter) | 10 |
| Total | 100 |

Ingredients were combined in a manner similar to that discussed above. The resulting mixture had a soft texture (with no grittiness), good glide, feels silky and smooth. The mixture stayed creamy white in an accelerated stability incubator of temperature 45° C. for 3 months.

The reduced ratio of cyclomethicome and crosspolymers results in higher viscosity and gives a thicker cream.

This system can be used in anti-wrinkle and collagen boosting preparation where easy to oxidize, watersoluble active Vitamin C can be incorporated for stability while avoiding the low pH problem (oxidation) that Vitamin C normally has.

### Example 6

### 12% Ascorbic Acid, Retinol

TABLE 9
Example 6

| Ingredient | % wt |
|---|---|
| Cyclomethicone (ie. Cyclopentasiloxane) | 40 |
| Ascorbic Acid | 12 |
| Dimethicone/Vinyl Dimethicone Crosspolymer | 10 |
| Dimethicone Crosspolymer | 20 |
| Silica Dimethyl Silylate | 3 |
| Oil (ie. Safflower oil) | 4.8 |
| Lipids (ie Shea Butter) | 10 |
| Retinol | 0.2 |
| Total | 100 |

Ingredients were combined in a manner similar to that discussed above. The resulting mixture had a soft texture (with no grittiness), good glide, feels silky and smooth. The mixture stayed creamy white in an accelerated stability incubator of temperature 45° C. for 3 months.

This Vitamin C system can be used in skin lightening preparation where easy to oxidize retinol (or Vitamin A) and Vitamin C can be combined without destabilizing each other. For instance, to be stable retinol requires a neutral pH of 6-7. Vitamin C has a pH around 2 in a normal, water-based preparation. Thus, the conditions to promote stability for the retinol destabilize Vitamin C and vice versa.

### Example 7

### Sunscreen and 12% Ascorbic Acid

TABLE 10
Example 7

| Ingredient | % wt |
|---|---|
| Cyclomethicone (ie. Cyclopentasiloxane) | 41.8 |
| Ascorbic Acid | 12 |
| Dimethicone/Vinyl Dimethicone Crosspolymer | 10 |
| Dimethicone Crosspolymer | 20 |
| Silica Dimethyl Silylate | 3 |
| Liquid Oil (ie. Safflower oil) | 0.2 |
| Hydrogeneted Vegetable Butter | 5 |
| Zine Oxide | 3 |
| Titanium Dioxide | 5 |
| Total | 100 |

Ingredients were combined in a manner similar to that discussed above. The resulting mixture had a soft texture (with no grittiness), good glide, feels silky and smooth. The mixture stayed creamy white in an accelerated stability incubator of temperature 45° C. for 3 months.

This Vitamin C system can be used in a sunscreen application into which hard to disperse inorganic sunscreen like zinc oxide and/or titanium oxide can be incorporated while avoiding coagulation of the mineral particles. Both zinc oxide and titanium oxide require a neutral pH of 7 to be stable while vitamin C generates an acid pH of around 2 in a normal water-based preparation.

### Example 8

### Benzyl Peroxide

TABLE 11
Example 8

| Ingredient | % wt |
|---|---|
| Cyclomethicone (ie. Cyclopentasiloxane) | 46.5 |
| Ascorbic Acid | 20.00 |
| Dimethicone/Vinyl Dimethicone Crosspolymer | 3.00 |
| Dimethicone Crosspolymer | 20.00 |
| Sillica Dimethyl Silylate | 0.50 |
| Oil (ie. Safflower oil) | 5.00 |
| Lipids (ie Shea Butter) | 2.00 |
| Benzyl peroxide | 3.00 |
| Total | 100.00 |

Ingredients were combined in a manner similar to that discussed above. The resulting mixture had a soft texture (with no grittiness), good glide, feels silky and smooth. The mixture stayed creamy white in an accelerated stability incubator of temperature 45° C. for 3 months.

This Vitamin C system can be used for acne-prone skin treatment where unstable and hard to solubilize active benzyl peroxide can be incorporated to avoid the oxidizing degradation. Additionally the anhydrous preparation itself inhibits the growth of the acne-causing bacteria.

### Example 9

### 10% Ascorbic Acid and Minoxidil

TABLE 12
Example 9

| Ingredient | % wt |
|---|---|
| Cyclomethicone (ie. Cyclopentasiloxane) | 56.3 |
| Ascorbic Acid | 10.0 |
| Dimethicone/Vinyl Dimethicone Crosspolymer | 2.0 |
| Dimethicone Crosspolymer | 20.0 |
| Sillica Dimethyl Silylate | 3.0 |
| Liquid Oil (ie. Safflower oil) | 3.0 |
| Hydrogeneted Vegetable Oil | 0.2 |
| Phytostrols | 0.5 |
| Minoxidil | 5.0 |
| Total | 100.0 |

Ingredients were combined in a manner similar to that discussed above. The resulting mixture had a soft texture (with no grittiness), good glide, feels silky and smooth. The mixture stayed creamy white in an accelerated stability incubator of temperature 45° C. for 3 months.

The system can be used for hair loss prevention wherein hard-to-soubilize active ingredients like phytosterois and/or minoxidil are incorporated.

### Example 10

### 20% Ascorbic Acid and Stem Cell Culture Extract

TABLE 13
Example 10

| Ingredient | % wt |
|---|---|
| Cyclomethicone (ie. Cyclopentasiloxane) | 41 |
| Ascorbic Acid | 20 |
| Dimethicone/Vinyl Dimethicone Crosspolymer | 2 |
| Dimethicone Crosspolymer | 11 |
| Sillica | 5 |
| Liquid Oil (ie. Safflower oil) | 5 |
| Hydrogenated Vegetable Butter | 10.5 |
| Phytosterols | 0.5 |
| Stem cell culture extract | 5 |
| Total | 100 |

Ingredients were combined in a manner similar to that discussed above. The resulting mixture had a soft texture (with no grittiness), good glide, feels silky and smooth. The mixture stayed creamy white in an accelerated stability incubator of temperature 45° C. for 3 months.

This Vitamin C system can be used for water-free wound-healing preparation where water soluble anti-inflammatory active ingredients such as stem cell extracts and/or tissue respiratory factor can be incorporated. Additionally the anhydrous preparation itself prevents wound infection by inhibiting the growth of bacteria.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The spirit and scope of the appended claims should not be limited to the description of the preferred embodiments contained herein.

1. A substantially anhydrous composition comprising:
(a) about 1-40% by weight powdered L-ascorbic acid, or L-ascorbic acid derivatized at the C2, C3, C5, or C6 position;
(b) one or more crosspolymers and/or oleosomes as natural substitute for encapsulating the L-ascorbic acid,
(c) at least one medium to high melting lipid and/or at least one low melting point lipid; and
(d) one or more volatile compounds as dispersing agent for L-ascorbic acid.

2. The substantially anhydrous composition according to claim 1, wherein the powdered L-ascorbic acid derivatized at the C2, C3, C5, or C6 position is water-soluble.

3. The substantially anhydrous composition according to claim 1 wherein the L-ascorbic acid derivative is ethyl-ascorbic acid, ascorbyl-2-glucoside (ASC-G), ascorbyl-6-octanoate (ASC-8), ascorbyl-6-palmitate (ASC-P), ascorbyl-6-stearate (ASC-S), ascorbyl-2,6-dipalmitate (ASC-DP), L-dehydroascrobic acid, sodium ascorbyl phosphate, ascorbyl tetraisopalmitate/tetrahexyldecyl ascorbate, or magnesium ascorbyl phosphate.

4. The substantially anhydrous composition according to claim 1, comprising about 10-30% by weight L-ascorbic acid, or L-ascorbic acid derivatized at the C2, C3, C5, or C6 position.

5. The substantially anhydrous composition according to claim 1, wherein the one or more crosspolymers are selected from the group consisting of:
acrylates/bis-hydroxypropyl dimethicone crosspolymer, behenyl dimethicone/bis-vinyldimethicone crosspolymer; bis-phenylisopropyl phenylisopropyl dimethicone/vinyl dimethicone crosspolymer; bis-vinyldimethicone/bis-isobutyl PPG-20 crosspolymer; bis-vinyldimethicone crosspolymer; bis-vinyldimethicone/l PEG-10 dimethicone crosspolymer; bis-vinyldimethicone/PPG-20 crosspolymer; butyldimethicone methacrylate/methyl methacrylate crosspolymer; C30-45 alkyl cetearyl dimethicone crosspolymer; C4-24 alkyl dimethicone/divinyldimethicone crosspolymer; C30-45 alkyl dimethicone/polycyclohexene oxide crosspolymer; cetearyl dimethicone crosspolymer; cetearyl dimethicone/vinyl dimethicone crosspolymer; cetyl dimethicone/bis-vinyldimethicone crosspolymer; cetyl hexacosyl dimethicone/bis-vinyldimethicone crosspolymer; crotonic acid/vinyl C8-12 isoalkyl esters/VA/bis-vinyldimethicone crosspolymer; dimethicone/bis-isobutyl PPG-20 crosspolymer; dimethicone/bis-vinyldimethicone/silsesquioxane crosspolymer; dimethicone crosspolymer; dimethicone crosspolymer-3; dimethicone/divinyldimethicone/silsesquioxane crosspolymer; dimethicone/lauryl dimethicone/bis-vinyldimethicone crosspolymer; dimethicone/PEG-10 crosspolymer; dimethicone/PEG-10/15 crosspolymer; dimethicone/PEG-15 crosspolymer; dimethicone/phenyl vinyl dimethicone crosspolymer; dimethicone/polyglycerin-3 crosspolymer; dimethicone/PPG-20 crosspolymer; dimethicone/titanate crosspolymer; dimethicone/vinyl dimethicone crosspolymer; dimethicone/vinyltrimethylsiloxysilicate crosspolymer; diphenyl dimethicone crosspolymer; diphenyl dimethicone/vinyl diphenyl dimethicone/silsesquioxane crosspolymer; divinyldimethicone/dimethicone crosspolymer; hydroxypropyl dimethicone/polysorbate 20 crosspolymer; isopropyl titanium triisostearate/triethoxysilylethyl polydimethylsiloxyethyl dimethicone crosspolymer; lauryl dimethicone PEG-15 crosspolymer; lauryl dimethicone/polyglycerin-3 crosspolymer; lauryl polydimethylsiloxyethyl dimethicone/bis-vinyldimethicone crosspolymer; PEG-10 dimethicone crosspolymer; PEG-12 dimethicone crosspolymer; PEG-8 dimethicone/polysorbate 20 crosspolymer; PEG-12 dimethicone/bis-isobutyl PPG-20 crosspolymer; PEG-12 dimethicone/PPG-20 crosspolymer; PEG-10 dimethicone/vinyl dimethicone crosspolymer; PEG-10/lauryl dimethicone crosspolymer; PEG-15/lauryl dimethicone crosspolymer; PEG-15/lauryl polydimethylsiloxyethyl dimethicone crosspolymer; perfluorononyl dimethicone/methicone/amodimethicone crosspolymer; polydimethylsiloxyethyl dimethicone/bis-vinyldimethicone crosspolymer; polyglyceryl-3/lauryl polydimethylsiloxyethyl dimethicone crosspolymer; silicone quatemium-16/glycidoxy dimethicone crosspolymer; styrene/acrylates/dimethicone acrylate crosspolymer; trifluoropropyl dimethicone/PEG-10 crosspolymer; trifluoropropyl dimethicone/trifluoropropyl, divinyldimethicone crosspolymer; trifluoropropyl dimethicone/vinyl trifluoropropyl dimethicone/silsesquioxane crosspolymer; trimethylsiloxysilicate/dimethicone crosspolymer; vinyl dimethicone/lauryl/behenyl dimethicone crosspolymer; vinyl dimethicone/lauryl dimethicone crosspolymer; vinyl dimethicone/methicone silsesquioxane crosspolymer; vinyldimethyl/tnmethylsiloxysilicate/dimethicone crosspolymer; and vinyldimethyl/trimethylsiloxysilicate stearyl dimethicone crosspolymer.

6. The substantially anhydrous composition according to claim 1, wherein the amount of crosspolymer ranges from about 5% by weight to 60% by weight.

7. The substantially anhydrous composition according to claim 1, comprising a medium to high melting point lipid wherein the medium to high melting point lipid has a melting point ranging from about 30° C. to about 80° C.

8. The substantially anhydrous composition according to claim 7, wherein the medium to high melting lipid is selected from the group consisting of: shea butter, apricot butter, cocoa butter, mango butter, kokum butter, illipe butter, cupuacu butter, murumuru seed butter, acrocomia aculeata butter, astrocaryum tucuma butter, attalea maripa butter, bassia latifolia seed butter, camellia reticulate seed butter, carpotroche brasiliensis seed butter, coconut seed butter, dipteryx odorate seed butter, palm butter, strawberry seed butter, sunflower seed butter, grcinia indica seed butter, walnut seed butter, irvingia gabonensis kernel butter, sesame seed butter, avocado butter, pentadesma butyracea seed butter, jojoba butter, shorea robusta seed butter, Theobroma grandiflorum seed butter, trichilia seed butter, virola surinamensis seed butter, pouteria sapota seed butter, and tribehenin.

9. The substantially anhydrous composition according to claim 7, wherein the amount of medium to high melting point lipid ranges from 1% to 30%.

10. The substantially anhydrous composition according to claim 7, wherein the medium to high melting point lipid is a wax selected from one or more of the group consisting of: Rhus Succedanea Fruit Wax, Polianthes Tuberosa Flower Wax, Helianthus Annuus (Sunflower) Seed Wax, Rosa Multiflora Flower Wax, Citrus Aurantium Dulcis (Orange) Flower Wax, Narcissus Poeticus Flower Wax, Acacia Decurrens Flower Wax, Jasminum Grandiflorum (Jasmine) Flower Wax, Nelumbo Nucifera Flower Wax, Candelilla Cera, Cistus Ladaniferus Flower Wax, Citrus Aurantium Amara (Bitter Orange), Cananga Odorata Flower Wax, Citrus Limon (Lemon) Peel Wax, Copemicia Cerifera (Camauba) Wax, Copemicia Cerifera Cera, Euphorbia Cerifera (Candelilla) Wax, Cytisus Scoparius Flower Wax, Myrica Cerifera (Bayberry) Fruit Wax, Tuberose Wax, Palm Kernel Wax, Helichrysum Angustifolium Wax, Ericerus Pela Wax, Tribehenin, Beeswax, Lanolin Wax, and Petrolatum wax.

11. The substantially anhydrous composition according to claim 1, wherein the low melting point lipid is an oil selected from one or more of the group consisting of cyclopentasiloxane, olus oil, camelina sativa oil, vegetable oil, palm oil, camelina sativa seed oil, Abyssinian oil, apricot kernel oil, fractionated coconut oil, jojoba oil, mongongo nut oil, pine nut oil, cashew oil, beech nut oil, sweet almond oil, walnut oil, wheat germ oil, avocado oil, borage seed oil, cranberry seed oil, evening primrose oil, grape seed oil, hazelnut oil, kukui nut oil, macademia nut oil, meadowfoam oil, olive oil, peanut oil, pecan oil, pomegranate seed oil, rose hip oil, seabuckthom berry oil, sesame seed oil, sunflower oil, watermelon seed oil, canola oil, camellia oil, tigemut oil, thistle oil, safflower oil, oat oil, marula oil, Moroccan oil, dika oil, fish oil, kapok seed oil, peanut oil, seje oil, sacha inchi oil, palm kernel oil, kenaf seed oil, and argan oil.

12. The substantially anhydrous composition according to claim 7, wherein the composition comprises either a high melting point lipid or a low melting point lipid.

13. The substantially anhydrous composition according to claim 1, wherein the amount of volatile compound(s) ranges from 1% to 60% by weight.

14. The substantially anhydrous composition according to claim 1, wherein the volatile compound(s) is a silicon containing volatile oil or a hydrocarbon.

15. The substantially anhydrous composition according to claim 1, wherein the volatile compound(s) is selected from the group consisting of: diolyldimethylsiloxane, cyclopentylsiloxane, cyclomethicone, polymethylphenylsilxoxane, hydrocarbons, isododecane, undecane, tridecane, and coconut alkanes.

16. The substantially anhydrous composition according to claim 1, in the form of an oil, gel, paste, lotion, crème, or aerosolized oil, gel, lotion or crème.

17. The substantially anhydrous composition according to claim 11, wherein the composition has a low melting point lipid and is in the form of a gel.

18. The substantially anhydrous composition of claim 1, wherein the composition further comprises an additional active ingredient.

19. The substantially anhydrous composition of claim 18, wherein the additional active ingredient is selected from an antiperspirant, a sunscreen, a yeast, a stem cell, or an antibiotic.

20. A method for treating skin wrinkles, for skin lightening, for increasing collagen production or retention, for moisturizing skin, for treating or alleviating acne, or for preventing sun damage comprising administering the composition of claim 1.
